(19) United States
(12) Patent Application Publication
(10) Pub. No.: US 2023/0210194 A1
(43) Pub. Date: Jul. 6, 2023

(54) POSTPARTUM BELLY WRAP

(71) Applicant: Lizeth Cuara, Mission Hills, CA (US)

(72) Inventor: Lizeth Cuara, Mission Hills, CA (US)

(21) Appl. No.: 18/114,237

(22) Filed: Feb. 25, 2023

(51) Int. Cl. A41C 1/10; A61F 7/02; A61F 5/02

(52) U.S. Cl. CPC A41C 1/10; A61F 7/02; A61F 5/028; A61F 2007/023; A61F 2007/0022

(57) ABSTRACT

Presented is a belly wrap wearable by a postpartum woman The belly wrap includes a wrap body having a first side, and a second side. The wrap body includes a back support section with a pocket disposed thereof, a first side section, and a second side section attached on either side of the back support section, and an abdomen support section attached to one side of the second side section. The abdomen support section includes a pocket configured on the first side of the wrap body, and a first matting means configured on the second side of the wrap body. The first side section further includes finger-like projections extending therefrom. The finger-like projections include a head incorporating a second mating means. The second mating means removably engages with the first mating means to ensure the belly wrap is wrapped around the abdomen and back of the woman.

### TECHNICAL FIELD OF INVENTION

The present invention relates to abdominal supports, and more particularly to a belly wrap wearable by a postpartum woman to stabilize their core muscles and tissue, restore scars/stretch marks women usually gain post-pregnancy, and help women receive heat and cold therapy for added comfort.

### BACKGROUND

After giving a birth, a woman's abdomen is generally extended and it requires some time for the body to recover and return to a normal size. Also, most women suffer lower back pain for a long period of time after delivery. Various abdominal supports are well-known and are typically designed to provide support and stability to a user's abdomen and back. Conventional abdominal supports are often present in the form of a belt or wrap, which is wrapped around a user's abdomen and secured to itself. Such supports are often bulky and uncomfortable to wear and fail to provide a proper supportive fit. Also, such supports are not made in a manner to be functional and comfortable for extended wear over many hours.

In the past, stretchable garments are provided as a solution to such problems that can be worn by a woman during pregnancy (Eg. STRETCHEAZ™ is one of such products available in the market by Holmes Fraser, Pa.) or during body's postpartum phase that would assist in shrinking abdomen and back to normal size while being comfortable. The stretchable garments are made from a stretchable material that tightly conforms to the body. The degree of stretchability is dependent on the wearer's individual body shape and size and the garment. Stretchable garments are used by postpartum women to support the back and abdominal muscles during pregnancy after pregnancy, offering the new mother comfort.

Further, Diastasis recti (hereinafter “DR”) related to a separation of the abdominal muscles associated with women (especially after childbirth) is increasingly becoming a challenge to overcome. If left untreated, DR can result in various medical problems, some of which could be disabling, including hernias, lower back pain, and other complications.

Although solutions exist in the past for providing support and stability to a user's abdomen and back during pregnancy and/or during the postpartum phase, they have their own limitations. Thus, the inventor herein proposes a novel postpartum belly wrap. The wearable postpartum belly wrap of the proposed invention would prove to be better result oriented, comfort giving, when worn by women during their postpartum phase.

### SUMMARY

Accordingly, there is provided a postpartum belly wrap wearable by women that helps stabilize core muscles and tissue of the abdomen region and lower back of the women,

Another objective of the present invention is to provide a postpartum belly wrap usable by a woman to heal and restore the elasticity of the skin over C-section scars and stretch marks of the abdomen area and the lower back.

Another objective of the present invention is to provide a belly wrap for postpartum women that comprises interior pockets that can be used to hold heat and ice packs to provide heat and cold therapy post birth giving.

Another objective of the present invention is to provide a belly wrap for postpartum women that helps with DR by aiding the closure of the abdominal gap after pregnancy.

Embodiments of the present invention disclose a belly wrap wearable by a postpartum woman The belly wrap includes a wrap body having a first side configured to touch the abdomen and back of the woman's body, and a second side that does not touch the abdomen and back of the woman's body.

According to the embodiment, the wrap body comprising a back support section comprising a first pocket disposed on the first side thereof; a first side section, and a second side section, wherein the first side section and the second side section are continuously formed or attached on either side of the back support section; an abdomen support section continuously formed or attached to one side of the second side section, the abdomen support section comprising a second pocket configured on the first side thereof, and comprises a first matting means configured on the second side of the wrap body.

According to the embodiment, the first side section further comprising a plurality of finger-like projections extending therefrom, each of the plurality of finger-like projections comprising a head incorporating a second mating means.

These and other features and advantages along with other embodiments of the present invention will become apparent from the detailed description below, in light of the accompanying drawings.

### BRIEF DESCRIPTION OF DRAWINGS

In the drawings:

FIG. 1 illustrates a perspective view of a belly wrap wearable by a postpartum woman, according to an exemplary embodiment of the present invention.

FIG. 2 is a plan view of the postpartum belly wrap of FIG. 1, showing the side that faces the body of the woman

FIG. 3 is a plan view of the postpartum belly wrap of FIG. 1, showing the front face side, which does not touch the abdomen or back of the woman.

FIG. 4 is a perspective view of the postpartum belly wrap of FIG. 1 where the postpartum belly wrap is in a rolled-up state.

FIG. 5 shows the postpartum belly wrap of FIG. 1 in a partially worn state by a woman.

FIG. 6 shows the postpartum belly wrap of FIG. 1 in a fully worn state by a woman

### DETAILED DESCRIPTION

Some embodiments, illustrating its features, will now be discussed in detail. The words “comprising,” “having,” “containing,” “including,” “consisting,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments in description and drawings are merely exemplary.

References to “one embodiment”, “an embodiment”, “another embodiment”, “an example”, “another example”, “some embodiment”, “yet another embodiment”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. Unless stated otherwise, terms such as “first”, “second”, “third”, are used to arbitrarily distinguish between the elements such terms describe. Thus, these terms are not necessarily intended to indicate temporal or other prioritization of such elements.

Before describing the present invention in detail, it should be observed that the present invention relates to a postpartum belly wrap wearable by a woman. Accordingly, the components have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting but as a representative basis for teaching one skilled in the art to variously employ the present invention in virtually any appropriately detailed structure. Further, the terms and phrases used herein are not intended to be limiting but rather to provide an understandable description of the invention.

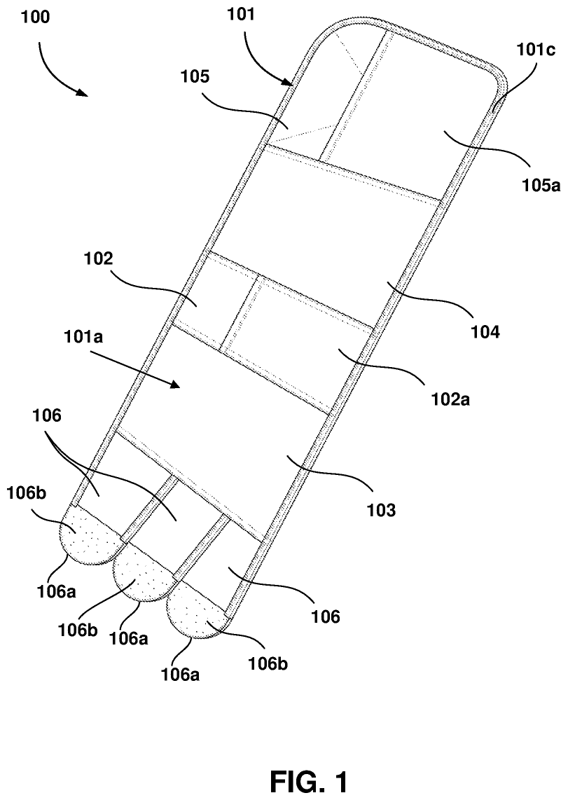

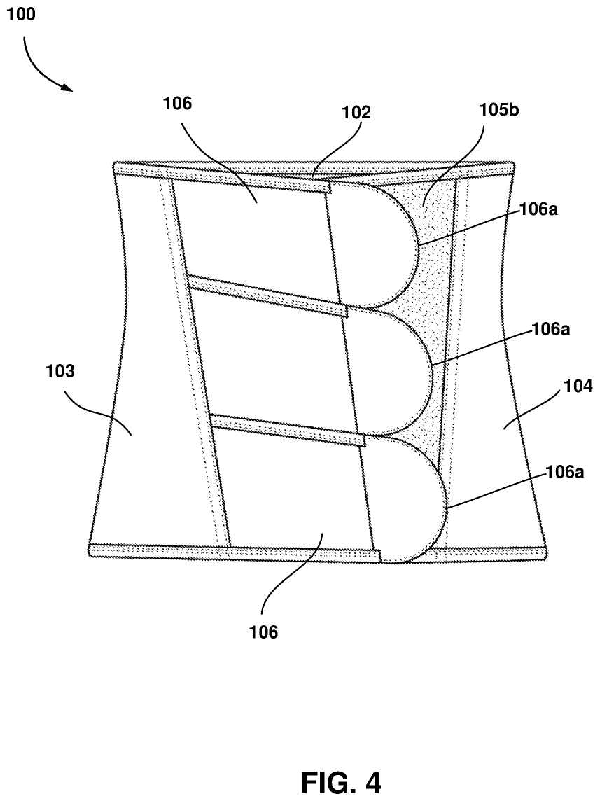

The various features and embodiments of the postpartum belly wrap wearable by a woman are better explained in conjunction with FIGS. 1-6.

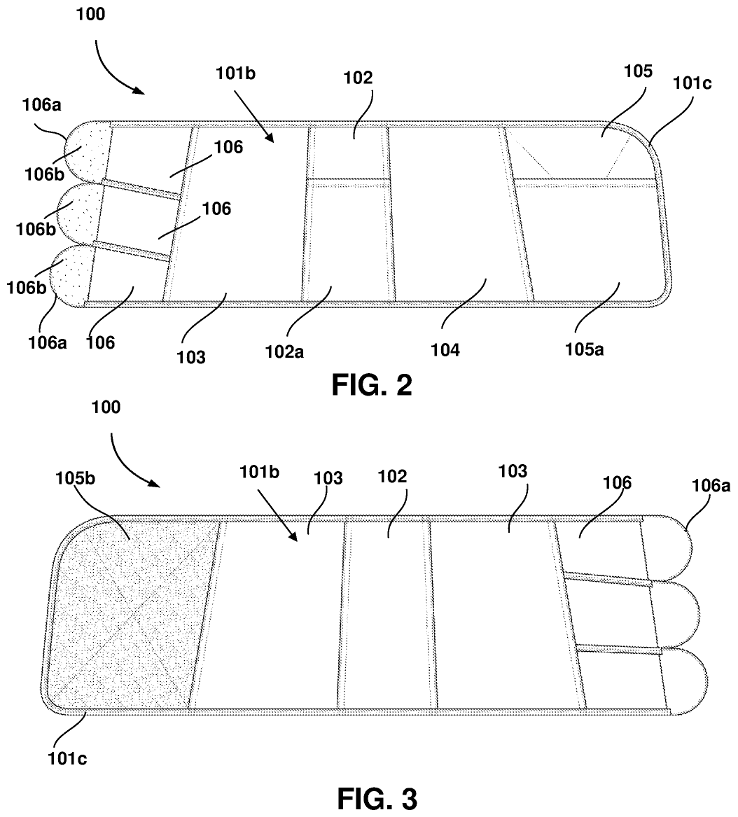

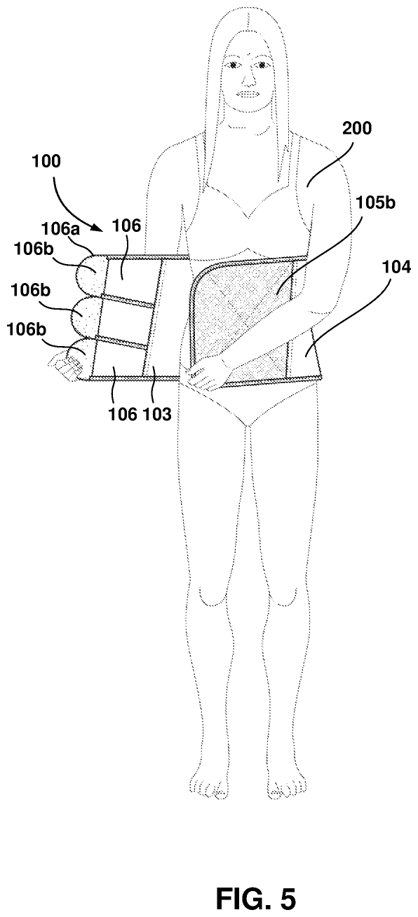

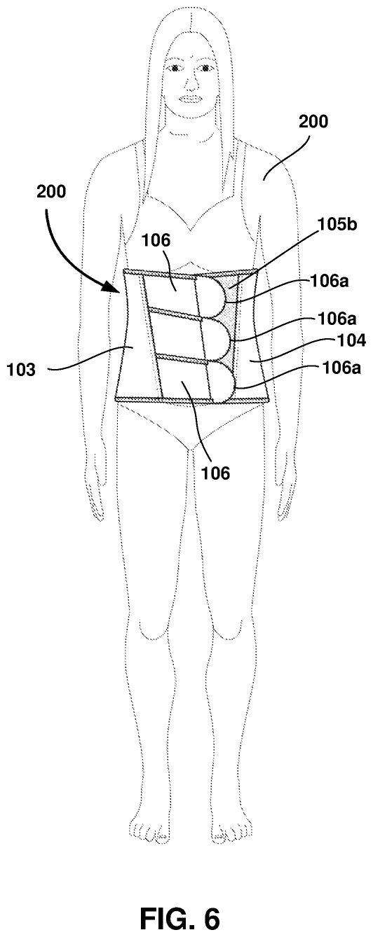

Referring to the accompanying figures, particularly FIGS. 1-6, the postpartum belly wrap 100 (hereinafter referred to as “wrap 100”) of the present invention includes a wrap body 101. The wrap body 101 includes a first side 101a that touches the abdomen and back of the woman's body as seen in FIGS. 2 and 5. The wrap body 101 includes a second side 101b that does not touch the abdomen and back of the woman's body as seen in FIGS. 3 and 6. In an embodiment, the wrap body 101 is substantially rectangular in shape and may be suitably sized so that the wrap 100 may be worn by number of women having a differently sized body.

The first side 101a of the wrap body 101 includes a first fabric material, and the second side 101b of the wrap body 101 includes a second fabric material. In one embodiment, the first fabric material and the second fabric material may be the same/identical material. In another embodiment, the first fabric material and the second fabric material may be dissimilar/unidentical. The first fabric material and the second fabric material may include but are not limited to cotton, nylon, polyester, elastic, netted fabric, non-netted fabric, and leather. The material may be any other suitable material including sweat-resistant material. The first and second fabric material of the first side a01a and the second side 101b of the wrap body 101 is stitched using a tape cloth 101c as seen in the accompanying figures. In another embodiment, the first and second fabric material of the first side a01a and the second side 101b of the wrap body 101 may be directly stitched by sewing.

Referring to FIGS. 1-4, the wrap body 101 includes a back support section 102 substantially rectangular in shape. The back support section 102 includes a first pocket 102a disposed on the first side 101a of the wrap body 101. The pocket 102a as seen is stitched (sewn) at three sides with respect to the first side 101a or the back support section 102. The pocket 102a is left unstitched at one side to form an opening for receiving hot and cold gel packs inside the pocket 102a. The hot and cold gel pack received within the pocket 102 provide heat and cold therapy to the back of the postpartum woman 200 and helps is relieving pain. In one embodiment, the pocket 102a may be sized smaller than the size of the back support section 102 as seen in FIGS. 1 and 2. In another embodiment, the pocket 102a may be sized as the size of the back support section 102 (not seen). The first pocket 102a may be formed using a material selected from a group of materials consisting of but not limited to cotton, nylon, polyester, elastic, netted fabric, non-netted fabric, and leather.

The wrap body 101 further includes a first side section 103, and a second side section 104. The first side section 103 and the second side section 104 are continually formed or separately attached on either side of the back support section 102. The first and second sections 103,104 are substantially rectangular in shape and preferably identically sized. In some embodiment, the first and second sections 103,104 and the back support section 102 may be identically sized. In some alternative embodiments, the first and second sections 103,104 and the back support section 102 may be differently sized.

The wrap body 101 further includes an abdomen support section 105. The abdomen support section 105 may be continuously formed or attached to one side of the second side section 104. The abdomen support section 105 includes a second pocket 105a configured on the first side 101a of the body wrap 101. The pocket 105a as seen is stitched (sewn) at three sides with respect to the first side 101a or the abdomen support section 105. The pocket 105a is left unstitched at one side to form an opening for receiving hot and cold gel packs inside the pocket 105a. The hot and cold gel pack received within the pocket 102 provides heat and cold therapy to the abdomen of the postpartum woman 200 and helps in overcoming C-section scars/stretched skin and also help mitigate DR problem in women that women usually have to suffer after giving birth to a child. In one embodiment, the pocket 105a may be sized smaller than the size of the abdomen support section 105 as seen in FIGS. 1 and 2. In another embodiment, the pocket 105a may be sized as the size of the abdomen support section 105 (not seen). The pocket 105a may be formed using a material selected from a group of materials consisting of but not limited to cotton, nylon, polyester, elastic, netted fabric, non-netted fabric, and leather.

The abdomen support section 105 further comprises a first matting means 105b configured on the second side 101b of the wrap body 101. The pocket 105a and the mating means 105b are opposingly disposed of on the abdomen support section 105 on either side of the wrap body 101.

The wrap body 101 further includes a plurality of finger-like projections 106. The plurality of finger-like projections 106 extends out from the side section 103. As seen, each of the plurality of finger-like projections 106 includes a head 106a incorporating a second mating means 106b. The head 106a of each of the finger like projections 106 is semi-circular shaped as shown in the accompanying figures according to a preferred embodiment of the present invention. However, it should be understood that, the head 106a may be square shaped , rectangular shaped, polygonal shaped. The mating means 106b is attached on an interior side of each of the heads 106a of the plurality of finger-like projections 106. The mating means 106b may be sized substantially same as the head 106b. The matting means 105b is made comparatively larger in size than the second mating means 106b to facilitate adjustability and comfort fit of the belly wrap 100 around the abdomen and back of the woman 200.

Further, in one embodiment, the mating means 105b,106b may be part of a hook and loop system similar to a VELCRO® fastener as shown in accompanying figures. In other embodiments, the mating means 105b,106b may be part of a hook and eye system, a button system, or a snap system. In yet another embodiments, the mating means 105b,106b may be an adhesive.

In operation, as shown in FIGS. 5 and 6, when the wrap 100 is worn by a user (woman 200), with the side 101a touching the body and wrapped around supporting the back and abdomen. The mating means 106b located on the finger-like projections 106 is removably engaged with the mating means 105b) located on the abdomen support section 105 to ensure the belly wrap 100 is wrapped around abdomen and back of the woman 200 and held in place for required time duration. When the wrap 100 is worn, the first pocket 102a and the second pocket 105a (holding the hot and cold ice packs) substantially conform to the back and abdomen of the postpartum woman 200 respectively to provide relief from back pain and stabilize core muscles and tissue of the abdomen region and lower back of the women. The worn wrap 100 also heals and restores the elasticity of the skin over C-section scars and stretch marks of the abdomen area. This also helps prevent DR by aiding the closure of the abdominal gap usually developed after pregnancy.

Further, although not discussed in the foregoing description, the wrap 100 may be a padded wrap to provide effective back support to the wearer. In an embodiment where the wrap is a padded wrap, the padding or cushioning or firm support substrate (not seen) is sandwiched between the first side 101a and the second side 101b of the wrap body 101 and may be stitched together with the sides 101a, 101b to keep the padding in place.

Although particular embodiments of the invention have been described in detail for purposes of illustration, various modifications and enhancements may be made without departing from the spirit and scope of the invention.

What is claimed is:

1. A belly wrap (100) wearable by a postpartum woman (200), comprising:
a wrap body (101) having a first side (101a) configured to touch abdomen and back of the woman's body, and a second side (101b) that does not touch the abdomen and back of the woman's body;
Wherein, the wrap body (101) comprising
a back support section (102) comprising a first pocket (102a) disposed on the first side (101a) thereof;
a first side section (103), and a second side section (104), wherein the first side section (103) and the second side section (104) are continuously formed or attached on either side of the back support section (102);
an abdomen support section (105) attached to one side of the second side section (104), the abdomen support section (105) comprising a second pocket (105a) configured on the first side (101a) thereof, and comprises a first matting means (105b) configured on the second side (101b) of the wrap body (101); and
wherein, the first side section (103) further comprising a plurality of finger-like projections (106) extending therefrom, each of the plurality of finger-like projections (106) comprising a head (106a) incorporating a second mating means (106b).

2. The belly wrap (100) of claim 1, wherein the first side (101a) of the wrap body (101) comprises a first fabric material, and the second side (101b) of the wrap body (101) comprises a second fabric material.

3. The belly wrap (100) of claim 2, wherein the first fabric material and the second fabric material are identical and selected from a group of materials consisting of cotton, nylon, polyester, elastic, netted fabric, non-netted fabric, and leather.

4. The belly wrap (100) of claim 2, wherein the first fabric material and the second fabric material are nonidentical and selected from a group of materials consisting of cotton, nylon, polyester, elastic, netted fabric, non-netted fabric, and leather.

5. The belly wrap (100) of claim 2, wherein the periphery of the first fabric material, and the second fabric material are stitched using a tape cloth or are directly stitched by sewing.

6. The belly wrap (100) of claim 1, wherein the second pocket (105a) and the first mating means (105b) are opposingly disposed of on the abdomen support section (105) on either side of the wrap body (101).

7. The belly wrap (100) of claim 1, wherein the head (106a) of each of the plurality of finger like projections (106) is semi-circular shaped.

8. The belly wrap (100) of claim 1, wherein the second mating means (106b) is attached on an interior side of each of the heads (106a) of the plurality of finger-like projections (106).

9. The belly wrap (100) of claim 1, wherein the second mating means (106b) located on the plurality of finger-like projections (106) removably engages with the first mating means (105b) located on the abdomen support section (105) to ensure the belly wrap (100) is wrapped around abdomen and back of the woman (200).

10. The belly wrap (100) of claim 1, wherein the first matting means (105b) is made comparatively larger in size than the second mating means (106b) to facilitate adjustability and comfort fit of the belly wrap (100) around the abdomen and back of the woman (200).

11. The belly wrap (100) of claim 1, wherein the first pocket (102a) and the second pocket (105a) are configured to removably receive a hot and cold gel pack therein to provide heat and cold therapy to the postpartum woman (200).

12. The belly wrap (100) of claim 1, wherein the first pocket (102a) and the second pocket (105a) substantially conform to the back and abdomen of the postpartum woman respectively when the belly wrap (100) is worn by the woman (200).

13. The belly wrap (100) of claim 1, wherein the first matting means (105b) and the second mating means (106b) is a VELCRO® fastener.

14. The belly wrap (100) of claim 1, wherein the first matting means (105b) and the second mating means (106b) is an adhesive.

15. The belly wrap (100) of claim 1, wherein the first pocket (102a) and the second pocket (105a) are formed on the back support section (102), and the abdomen support section (105) by sewing.

16. The belly wrap (100) of claim 15, wherein the first pocket (102a) and the second pocket (105a) are formed using a material selected from a group of materials consisting of cotton, nylon, polyester, elastic, netted fabric, non-netted fabric, and leather.
